(19) United States
(12) Patent Application Publication
(10) Pub. No.: US 2015/0290294 A1
(43) Pub. Date: Oct. 15, 2015

(54) METHODS FOR INHIBITING PLATELET AGGREGATION USING GLP-1 RECEPTOR AGONISTS

(71) Applicants: Mansoor HUSAIN, (US); Alison CAMERON-VENDRIG, (US); Heyu NI, (US)

(72) Inventors: Mansoor Husain, Toronto (CA); Alison Cameron-Vendrig, Ottawa (CA); Heyu Ni, North York (CA)

(21) Appl. No.: 14/440,227

(22) PCT Filed: Nov. 4, 2013
(86) PCT No.: PCT/CA2013/000939

§ 371 Date: May 1, 2015

Related U.S. Application Data
(60) Provisional application No. 61/721,819, filed on Nov. 2, 2012.

(51) Int. Cl. A61K 38/26; A61K 45/06; A61K 9/00

(52) U.S. Cl. CPC A61K 38/26; A61K 9/0019; A61K 45/06

(57) ABSTRACT

Methods for inhibiting platelet aggregation using glucagon-like peptide-1 receptor (GLP-1R) agonists are disclosed. GLP-1 receptors are demonstrated to be expressed in platelets and/or megakaryocytes. Activation of GLP-1 receptors by GLP-1 agonists, such as GLP-1 and exendin-4, inhibited thrombin-induced platelet aggregation.

### RELATED APPLICATIONS

This application claims priority to U.S. Provisional Patent Application No. 61/721,819 filed Nov. 2, 2012, the contents of which are incorporated by reference herein in their entirety.

### FIELD OF THE DISCLOSURE

The disclosure relates to methods for inhibiting platelet aggregation and more specifically to methods for inhibiting platelet aggregation using GLP-1Receptor agonists.

### BACKGROUND OF THE DISCLOSURE

Patients with Type 2 Diabetes (T2D) have an increased incidence of atherothrombotic cardiovascular events. Most anti-diabetic treatments, including insulin, have not been shown to reduce these cardiovascular event rates.

A meta-analysis studying the effects of more vs. less intensive glycemic control on cardiovascular events in patients with T2D found that more intensive glycemic control resulted in a modest reduction in CV events (Turnbull et al. 2009). In contrast, studies of patients treated with glucagon-like peptide-1 (GLP-1) targeted therapies show a much greater decrease in CV event incidence over a shorter study duration (Monami et al. 2010; Monami et al. 2011). Drugs that target Glucagon-Like Peptide-1 (GLP-1) are used to treat patients with T2D because of their ability to stimulate glucose-dependent insulin secretion from pancreatic beta cells.

GLP-1 is an incretin hormone secreted by intestinal L-cells, which are located mainly in the distal ileum and the colon. GLP-1 is produced by post-translational processing of the proglucagon prohormone. Prohormone convertase ⅓ (PC1/3) is responsible for cleaving GLP-1 from proglucagon. At all stages of processing, GLP-1 can be found in a glycine extended form, and in a C-terminal amidated form. PC1/3 initially cleaves proglucagon to form GLP-1(1-37), which is then amidated by α-amidating enzyme to become GLP-1(1-36)^{NH2}. These forms of GLP-1 are secreted in small amounts and can exert insulinotropic actions, however the majority of GLP-1 is further truncated to GLP-1(7-37) and GLP-1(7-36)^{NH2}. Once secreted, GLP-1 is rapidly degraded by dipeptidyl peptidase-4 (DPP-IV), which cleaves two amino acids at the GLP-1 N terminus (His-Ala) to produce the GLP-1 metabolite, GLP-1(9-36)^{NH2}.

DPP-IV (also known as CD26) was first identified for its role in the immune system where it is involved in the co-stimulatory signal necessary for the activation of T-cells, as well as signal transduction in the T-cell leading to cytokine production and proliferation. Gupta et al. (2012) recently reported that sitagliptin, a selective DPP-IV inhibitor, exhibited anti-platelet activity associated with an inhibitory effect on intracellular free calcium and tyrosine phosphorylation. However, recent data suggests that treatment of patients with diabetes at high risk of cardiovascular disease with DPP-IV inhibitors may increase the risk of hospitalization from heart failure (Scirica et al. 2013). Relatively little is known about the possible effects of incretin hormones such as GLP-1 and the activation of the GLP-1 receptor on platelet aggregation.

### SUMMARY OF THE DISCLOSURE

It has surprisingly been determined that human megakaryocytes express the GLP-1 receptor (GLP-1R) and that GLP-1R agonists are useful for inhibiting platelet aggregation. GLP-1R agonists including GLP-1(7-36) (SEQ ID NO: 3), the metabolite GLP-1(9-36) (SEQ ID NO: 4) and the GLP-1 analog Exendin-4 (SEQ ID NO: 5) were shown to inhibit thrombin induced platelet aggregation. Treatment of megakaryocyte cells with a GLP-1R agonist was also shown to result in an increase in intracellular cAMP levels. GLP-1R agonists are therefore also expected to be useful for the prevention or treatment of thrombotic cerebrovascular or cardiovascular disease.

Accordingly, in one aspect there is provided a method for inhibiting platelet aggregation comprising activating the Glucagon-Like Peptide-1 Receptor (GLP-1R). In one embodiment, the method comprises contacting one or more platelets and/or megakaryocytes with a GLP-1R agonist. In one embodiment, the platelets are contacted with the GLP-1R agonist in vitro, in vivo or ex vivo.

In one embodiment there is provided a method for inhibiting platelet aggregation in a subject comprising administering to the subject a GLP-1R agonist. Also provided in the use of a GLP-1R agonist for inhibiting platelet aggregation in a subject in need thereof. Also provided is a method for inhibiting platelet aggregation in a subject, the method comprising activating GLP-1R. In one embodiment, the GLP-1R is activated on platelets and/or megakaryocytes. In one embodiment, there is provided a GLP-1R agonist for use in inhibiting platelet aggregation in a subject in need thereof. Also provided is the use of a GLP-1R agonist in the manufacture of a medicament or a pharmaceutical composition for inhibiting platelet aggregation.

In one embodiment, the GLP-1R agonist is exogenous GLP-1, such as recombinant or synthetic GLP-1(7-36) or GLP-1(9-36) or the C-terminal amidated forms GLP-1(7-36)^{NH2} or GLP-1(9-36)^{NH2}. Optionally, the GLP-1R agonist is a GLP-1 analog such as exenatide. In one embodiment, the GLP-1R agonist is selected from exenatide, liraglutide, albigiutide, taspoglutide and lixisenatide.

In one embodiment, the GLP-1R agonist binds to GLP-1R on platelets or megakaryocytes. Optionally, the GLP-1R agonist increases intracellular cAMP levels in megakaryocytes and/or platelets.

In one embodiment, a GLP-1R agonist is administered to a subject or used in order to inhibit platelet aggregation. In one embodiment, the subject has, or is suspected of having, cardiovascular disease or is at risk of a thrombotic event. In one embodiment, the subject has not been diagnosed with Type 2 diabetes. In one embodiment, the subject has, or is suspected of having, Type 2 diabetes. In one embodiment, GLP-1R agonists are used or administered to inhibit platelet aggregation for the treatment or prevention of thrombosis. In one embodiment, GLP-1R agonists are used or administered to inhibit platelet aggregation for the treatment or prevention of thrombotic cardiovascular or cerebrovascular events in a subject in need thereof. For example, in one embodiment, the GLP-1R agonists are used or administered to a subject in response to a thrombotic event such as myocardial infarction or stroke. In one embodiment, the GLP-1R agonists are used or administered to a subject to inhibit platelet aggregation for the treatment or prevention of an atherothrombotic event.

In one embodiment, the methods and uses described herein are for inhibiting platelet aggregation in a subject with a high risk of thrombosis, such as a subject with atrial fibrillation, or a subject with diffuse coronary, cerebral or peripheral vascular disease. In one embodiment the subject has chronic renal failure and/or is on dialysis.

In one embodiment, the methods and uses described herein are for inhibiting platelet aggregation in a subject intolerant or unresponsive to other antiplatelet agents such aspirin, ADP receptor antagonists, or thrombin inhibitors.

In one embodiment, the methods and uses described herein are for inhibiting platelet aggregation in a subject with diabetes, obesity and/or hypertension.

In one embodiment, the methods and uses described herein are for inhibiting platelet aggregation in a subject who is experiencing a thrombotic event such as a myocardial infarction or stroke. In one embodiment, the methods and uses described herein are for inhibiting platelet aggregation prophylactically such as to prevent recurrence of atherothrombosis or a thrombotic event. For example, in one embodiment the GLP1-R agonists described herein are used in or administered to a subject who has previously had a thrombotic event such as a myocardial infarction or stroke.

The GLP-1R agonist described herein may be administered, used or formulated for use, as known in the art. For example, in one embodiment the GLP-1R agonist is formulated and/or administered as a pharmaceutical composition with pharmaceutically acceptable carrier or diluent. In one embodiment, the GLP-1R agonist is formulated for use by injection. In one embodiment, the GLP-1R agonist is conjugated to another molecule to improve the therapeutic or pharmacokinetic properties of the peptide, such as by pegylation.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

One or more embodiments of the disclosure will now be described in relation to the drawings in which:

FIG. 1 shows that incubation of human gel-filtered platelets with GLP-1(7-36) (100 pM-1 uM) for 15 min at 37° C. delayed and reduced thrombin (0.125 U)-induced human platelet aggregation. FIG. 1A shows representative traces of the effect of different doses of GLP-1 on platelet aggregation. FIGS. 1B-F show the effects of each dose separately compared to the same PBS control.

FIG. 2 shows that incubation of human gel-filtered platelets for 15 min at 37° C. with GLP-1(7-36) and GLP-1(9-36) (10 nM each) delayed human platelet aggregation after stimulation with 0.5 U thrombin. The effect is greater with GLP-1(7-36) than with GLP-1(9-36).

FIG. 3A shows that incubation with GLP-1 (1 nM) resulted in a 60% decrease in aggregation 1 min after thrombin stimulation (0.125 U). The dose-response curve appears J-shaped. The level of aggregation of 1 nM GLP-1(7-36)-treated platelets is significantly lower than control (n=3, p<0.5; one-way Anova and Dunnett's Multiple Comparison test for post hoc analysis). FIG. 3B shows that while not significant, the analysis of the total area under curve of the GLP-1-treated platelets shows that there appears to be some inhibition of platelet activity between 0.1 nM and 100 nM (n=3). Statistics were performed by one-way ANOVA and post-hoc analysis with Dunnett's Multiple Comparison Test. (*) indicates p<0.05.

FIG. 4 shows that incubation of human gel-filtered platelets with Exenedin-4 (0.1 nM-100 nM) for 15 min at 37° C. reduced and delayed thrombin (0.5 U)-induced platelet aggregation in a dose-dependent manner relative to PBS-treated controls. FIG. 4A shows representative traces of the effect of different doses of Exenedin-4 on platelet aggregation. FIGS. 4B-E show the effects of each dose separately compared to the same PBS control.

FIG. 5 shows the effect of Exendin-4 on platelet aggregation. FIG. 5A shows that 1 min after stimulation with 0.25 U thrombin, Exendin-4 (1 nM) treated platelets are inhibited by ˜35% (n=3). FIG. 5B. 6 min after stimulation with 0.25 U thrombin, during the second wave of aggregation, 0.1-10 nM exendin-4 inhibited platelet aggregation (n=3). FIG. 5C. While not significant, the total area under curve of the exendin-4 treated platelets shows that there appears to be some inhibition of platelet activity between 1 nM and 100 nM. Statistics were performed by one-way ANOVA and post-hoc analysis with Dunnett's Multiple Comparison Test. (*) indicates p<0.05, (#) indicates p<0.01

FIG. 6 shows GLP-1R expression probed in human platelets and human megakaryocytes (MEG-01) by Western blot. The GLP-1R antibody (Santa Cruz), was used at a 1:1250 dilution. HRP-conjugated secondary antibody and ECL was used to detect the GLP-1R signal. GAPDH served as loading control.

FIG. 7 is a bar graph showing the relative GLP-1R expression in human platelets and human megakaryocytes (MEG-01) by Western blot as shown in FIG. 6.

FIG. 8 shows that the GLP-1R sequence obtained from MEG-01 cells (SEQ ID NO: 2) is identical to the known human GLP-1R (SEQ ID NO: 1). A SNP at the 20th base pair causes a known missense mutation in the 7th amino acid (Proline to Leucine). This SNP has been previously described.

FIG. 9 shows that intracellular cAMP increases in response to GLP-1 and the GLP-1 receptor agonist exendin-4. The maximal response with both is approximately 2 fold relative to PBS control. FIG. 9A shows that incubation for 15 min with GLP-1 induces a dose-dependent increase in cAMP. (n=3) FIG. 9B shows that incubation for 15 minutes with exendin-4 increases intracellular cAMP. This response becomes significant from PBS control at 10 nM and is slightly decreased at 100 nM. (n=3). Statistics were performed by one-way ANOVA and post-hoc analysis with Dunnett's Multiple Comparison Test. (*) indicates p<0.05, (#) indicates p<0.01

FIG. 10A shows DPP4 activity assessed in different human cell types using the CBA085 Innozyme™ DPP4 Immunocapture Activity Assay from Merck™ chemicals. Human platelets and megakaryocytes do not appear to exhibit any detectable levels of DPP4 activity. FIG. 10B shows that platelets and MEG-01 cells do not harbour significant levels of DPP-IV activity. DPP-IV activity in human plasma can be inhibited by incubation with the DPP-IV inhibitor MK626. Statistics were performed by one-way ANOVA and post-hoc analysis with Newman-Keuls method (n=3). (#) indicates p<0.01

FIG. 11 shows that qRT-PCR results confirm the expression of GLP-1R mRNA in MEG-01 cells. MEG-01 GLP-1R expression levels are significantly less than in the pancreas, but significantly greater than in the lung. Expression levels were determined relative to the housekeeping gene TATA binding protein (TBP). Statistics were performed by one-way ANOVA and post-hoc analysis with Newman-Keuls method (n=3). (*) indicates p<0.05, (#) indicates p<0.01

FIG. 12A shows GLP-1R expression probed in human platelets and human and mouse megakaryocytes (MEG-01 and L8057) by Western blot. The GLP-1R antibody (Santa Cruz), was used at a 1:1250 dilution. HRP-conjugated secondary antibody and ECL was used to detect the GLP-1R signal. GAPDH served as loading control. FIG. 12B shows desitometry scans of the Western blot shown in FIG. 12A. Expression is highest in the MIN6 cells, followed by the human pancreas and L8057 cells. Human heart extract also shows considerable expression. MEG-01 and platelet extract expression is present, though much lower.

FIG. 13 shows that exendin-4 inhibits thrombus formation in an in vivo mouse cremaster ateriolar thrombosis model. FIG. 13A shows that thrombus formation is delayed and reduced after a single IV dose of 60 nmol/kg exendin-4. Control n=7, exendin-4 n=11. Images of the control (FIG. 13B) and treated thrombi (FIG. 13C) 50 seconds after laser injury. The thrombus appears significantly smaller after treatment with exenatide.

### DETAILED DESCRIPTION

The present description demonstrates that GLP-1, its metabolite, and agonists for the GLP-1 receptor (R) are anti-platelet agents and are useful for inhibiting platelet aggregation. GLP-1R agonists are known to be useful for the treatment of Type 2 Diabetes (T2D) given their ability to stimulate glucose-dependent insulin secretion from pancreatic beta cells. It has surprisingly been shown that megakaryocytes and platelets express GLP-1R and that GLP-1R agonists are useful for the inhibition of platelet aggregation irrespective of their anti-diabetic effects.

As shown in Example 1, native GLP-1, its metabolite, and exenatide, a commercially available GLP-1R agonist, are able to delay and inhibit thrombin-induced aggregation of human gel-filtered platelets. Furthermore, native GLP-1 has been shown to increase levels of intracellular cAMP in a human megakaryocyte cell line (MEG-01). As cAMP is an inhibitor of platelet function, this finding provides a molecular signaling mechanism underlying the inhibitory effect observed in aggregation experiments.

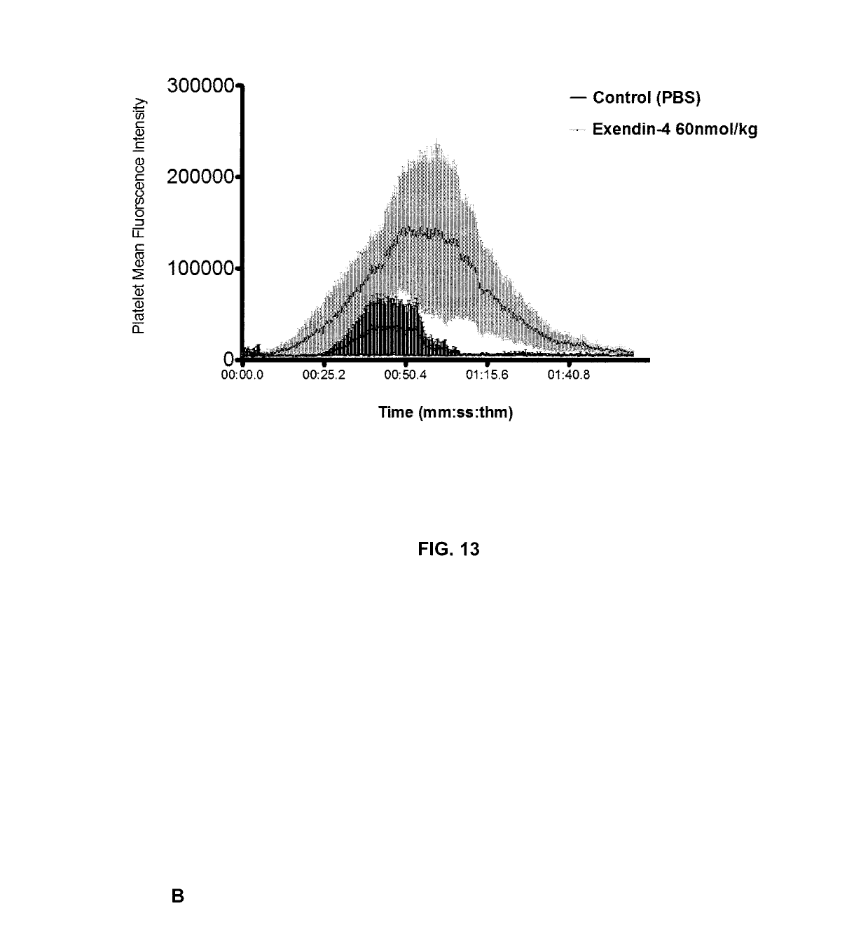

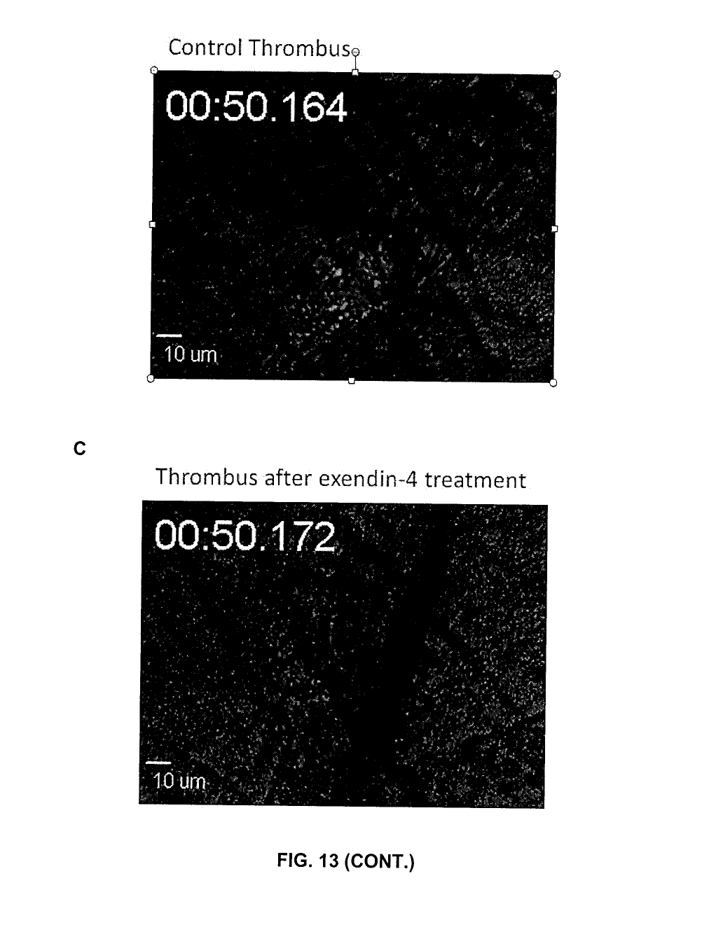

The GLP-1R is highly expressed on pancreatic beta cells, where its activation results in insulin secretion. As shown in Example 1 and in FIGS. 6-8, GLP-1R is also expressed on human platelets and megakaryocytes. It has also been determined that the endogenous ligand for this receptor, namely GLP-1, and an exogenous analog of GLP-1, namely exenatide, inhibit thrombin-induced platelet aggregation. Furthermore, the metabolite of GLP-1 known as GLP-1(9-36) has been demonstrated to have an inhibitory effect on platelet aggregation, albeit less potent than GLP-1(7-36). The GLP-1R agonist Exendin-4 was also confirmed to inhibit thrombus formation in an in vivo mouse cremaster arterioloar thrombosis model (FIG. 13 and Example 3). It has therefore surprisingly been shown that GLP-1R agonists are useful for inhibiting platelet aggregation. In one embodiment, GLP-1R agonists are useful for inhibiting platelet aggregation in subjects with cardiovascular disease. In one embodiment, GLP-1R agonists are used or administered to prevent platelet aggregation in a subject in need thereof, such as a subject at risk of a thrombotic event, optionally an atherothrombotic cardiovascular or cerebrovascular event.

Because T2D patients are at a high risk of clinically significant atherothrombotic cardiovascular events such as myocardial infarction, stroke and cardiovascular death, the anti-platelet effects of GLP-1R agonists described herein are particularly useful for reducing the risk of cardiovascular events in these patients. Furthermore, given that GLP-1R has been demonstrated to be expressed on megakaryocytes and platelets, GLP-1R agonists are useful as anti-platelet agents in subjects at risk of cardiovascular disease, regardless of their diabetic status.

Accordingly, in one embodiment there is provided a method for inhibiting platelet aggregation in a subject comprising administering to the subject a Glucagon-Like Peptide-1 Receptor (GLP-1R) agonist. Also provided is the use of a GLP-1R agonist for inhibiting platelet aggregation in a subject in need thereof. Also provided is a GLP-1R agonist for use in inhibiting platelet aggregation in a subject in need thereof.

As used herein, “subject” refers to any member of the animal kingdom, such as a mammal. In one embodiment the subject is a human. In one embodiment, the subject has a condition that would benefit from inhibiting platelet aggregation within the bloodstream. For example, in one embodiment, the subject has previously had a thrombotic event. In one embodiment, the subject has atherosclerotic plaques. In one embodiment, the subject has previously been administered or treated with an antiplatelet medication such as an adenosine diphosphate (ADP) receptor inhibitors such as clopidogrel, a phosphodiesterase inhibitor such as cilostazol, or a thromboxane inhibitor. In one embodiment, the subject does not have type 2 diabetes. In one embodiment, the subject has type 2 diabetes.

As used herein, “inhibiting platelet aggregation” refers to a reduction or delay in the aggregation of platelets. Platelets aggregate, or clump together, using fibrinogen and von Willebrand factor (vWF) as a connecting agent or optionally in the absence of fibrinogen and vWF (see for example Ni et al. 2000 and Dunne et al. 2012). Platelet aggregation can be triggered by injury to endothelial tissues and contact with thrombin. Optionally, inhibiting platelet aggregation also includes inhibiting thrombus formation. For example, antiplatelet drugs are commonly used for inhibiting platelet aggregation and for the prevention of thrombotic cerebrovascular or cardiovascular disease. As set out in Example 1, the inhibition of platelet aggregation can also be tested in vitro such as by using gel-filtered platelets, thrombin stimulation and an aggregometer.

As used herein “thrombosis” or a “thrombotic event” refers to the formation of a blood clot inside a blood vessel, obstructing the flow of blood through the circulatory system. Optionally, thrombosis results in ischemia. As used herein, “atherothrombosis” or an “atherothrombotic event” refers to atherosclerosis with superimposed platelet-rich thrombus formation, which obstructs the flow of blood through the circulatory system. Optionally, “atherothrombosis” or an “atherothrombotic event” results in an ischemic event such as a myocardial infarction or stroke.

As used herein “ischemia” or an “ischemic event” refers to any temporary or continuous blockage or restriction in blood flow to an organ or tissue causing a shortage of oxygen and/or glucose in the organ or tissue. Optionally, the term “ischemic event” includes a myocardial infarction, heart attack or stroke.

As used herein “type 2 diabetes” refers to a metabolic disorder characterized by high blood glucose in the context of insulin resistance and relative insulin deficiency condition. For example, in one embodiment a subject is considered to have type 2 diabetes if they have a glycated hemoglobin (A1C) level of 6.5 percent or higher on two separate tests. In one embodiment, a subject is considered to have type 2 diabetes if they have a fasting glucose levels of 126 mg/dL (7 mmol/L) or higher on two separate tests (see Definition and diagnosis of diabetes mellitus and intermediate hyperglycaemia, 2006 (ISBN: 978 92 4 159493 6) available from the World Health Organization, Diabetes Unit, Geneva, Switzerland).

As used herein, “Glucagon-Like Peptide-1 receptor (GLP-1R) agonist” refers to any compound or agent that activates GLP-1R. Examples of GLP-1R agonists include, but are not limited to GLP-1 polypeptides and/or metabolites such as GLP-1(7-36) (SEQ ID NO: 3) or GLP-1(9-36) (SEQ ID NO: 4) as well as GLP-1 analogs. In one embodiment, the GLP-1 analog is exendin-4 (SEQ ID NO: 5). Optionally, the GLP-1 polypeptides are amidated at the C-terminus i.e. GLP-1(7-36)^{NH2}. As used herein, the terms GLP-1(7-36) and GLP-1(9-36) include the amidated forms of the peptide. A peptide that is amidated at the C-terminus does not terminate with a carboxylic acid group, but rather with an amide group such as CONH₂. In one embodiment, the GLP-1R agonist is an exogenous GLP-1 polypeptide such as synthetic or recombinant GLP-1(7-36) or GLP-1(9-36). In one embodiment, the GLP-1R agonist is a fragment of a GLP-1R agonist polypeptide, such as a fragment of GLP-1 (7-36), GLP-1(9-36) or exendin-4 that binds to and activates GLP-1R. For example, in one embodiment the GLP-1R agonist comprises, consists essentially of, or consists of a fragment between 10-15, 15-20 or 20-29 amino acids of a known GLP-1R agonist polypeptide that binds to and activates GLP-1R. In one embodiment, the GLP-1R agonist is a fragment of between 10-15, 15-20 or 20-29 amino acids of exendin-4, GLP-1(7-36), GLP-1(9-36) or exendin-4. In one embodiment, the GLP-1R agonist is a fragment that increases cAMP levels in megakaryocytes or platelets. In one embodiment, the GLP-1R agonist is a polypeptide with sequence identity to a known GLP-1R agonist polypeptide, such as GLP-1(7-36), (GLP-1(9-36) or exendin-4. In one embodiment, the GLP-1R agonist has at least 70%, at least 80%, at least 90%, at least 95% sequence identity to the polypeptide sequence of GLP-1(7-36) (SEQ ID NO: 3), GLP-1(9-36) (SEQ ID NO: 4) or exendin-4 (SEQ ID NO: 5). In one embodiment, the GLP-1R agonist has sequence identity to a known GLP-1R agonist polypeptide and increases the levels of cAMP in megakaryocytes or platelets.

Sequence identity is typically assessed by the BLAST version 2.1 program advanced search (parameters as above; Altschul, S. F., Gish, W., Miller, W., Myers, E. W. & Lipman, D. J. (1990) “Basic local alignment search tool.” J. Mol. Biol. 215:403-410). BLAST is a series of programs that are available online through the U.S. National Center for Biotechnology Information (National Library of Medicine Building 38A Bethesda, Md. 20894) The advanced Blast search is set to default parameters. References for the Blast Programs include: Altschul, S. F., Gish, W., Miller, W., Myers, E. W. & Lipman, D. J. (1990) “Basic local alignment search tool.” J. Mol. Biol. 215:403-410; Gish, W. & States, D. J. (1993) “Identification of protein coding regions by database similarity search.” Nature Genet. 3:266-272; Madden, T. L., Tatusov, R. L. & Zhang, J. (1996) “Applications of network BLAST server” Meth. Enzymol. 266:131-141; Altschul, S. F., Madden, T. L., Schäffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402); Zhang, J. & Madden, T. L. (1997) “PowerBLAST: A new network BLAST application for interactive or automated sequence analysis and annotation.” Genome Res. 7:649-656).

Optionally, the GLP-1R agonist is a GLP-1 analog such as exenatide, liraglutide, albiglutide, taspoglutide and lixisenatide. The GLP-1 agonists described herein may also include naturally occurring GLP-1 analogs or synthetic GLP-1 analogs such as Exendin-4.

In one embodiment, the GLP-1R agonist is an analog of a known GLP-1R polypeptide agonist such as GLP-1. The analog is optionally a peptoid, which is an N-substituted polyglycine with amino acid R groups attached at the N atom. Another analog is optionally a peptide synthesized from D-amino acids rather than the natural L-amino acids. GLP-1R agonist peptides are readily prepared by chemical synthesis using techniques well known in the art related to the chemistry of proteins such as solid phase synthesis or by using recombinant techniques.

In one embodiment, the GLP-1R agonists described herein may be conjugated to another peptide or biomolecule in order to improve the pharmacokinetic or therapeutic qualities of the agonist. For example, in one embodiment the agonist is conjugated to a biologically compatible polymer such as polyethylene glycol (PEG) polymer. Optionally, the agonists are formulated for extended release or packaged in a suitable delivery vehicle.

In one embodiment, there is provided a pharmaceutical composition comprising a GLP-1R agonist as described herein and a pharmaceutically acceptable carrier or diluent. The GLP-1R agonists of the invention are optionally formulated into a pharmaceutical composition for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals. One aspect of the disclosure also includes the use of the GLP-1R agonists of the invention for preparation of a medicament or pharmaceutical composition for inhibiting platelet aggregation.

The GLP-1R agonists and/or pharmaceutical compositions comprising said GLP1-R agonists can be administered to humans or animals by a variety of methods including, but not restricted to topical administration, oral administration, aerosol administration, intratracheal instillation, intraperitoneal injection, injection into the cerebrospinal fluid, intravenous injection, intramuscular injection and subcutaneous injection. Dosages to be administered depend on patient needs, on the desired effect and on the chosen route of administration. In a preferred embodiment, the GLP-1R agonists described herein are administered into the bloodstream, such as by injection.

The pharmaceutical compositions can be prepared by known methods for the preparation of pharmaceutically acceptable compositions which can be administered to patients. In an embodiment, an effective quantity of the GLP-1R agonist is combined in a mixture with a pharmaceutically acceptable carrier. Suitable carriers are described, for example in Remington's Pharmaceutical Sciences (Remington's Pharmaceutical Sciences, Mack Publishing Company, Easton, Pa., USA) or Handbook of Pharmaceutical Additives (compiled by Michael and Irene Ash, Gower Publishing Limited, Aldershot, England (1995). On this basis, the compositions include, albeit not exclusively, solutions of the substances in association with one or more pharmaceutically acceptable carriers or diluents, and may be contained in buffered solutions with a suitable pH and/or be iso-osmotic with physiological fluids.

In one embodiment, a GLP-1R agonist as described herein is combined with other components such as a carrier in a composition such as a pharmaceutical composition. Optionally the GLP-1R agonists described herein may be combined in a pharmaceutical composition, used or co-administered with an anticoagulant, thrombolytic agent, anti-inflammatory and/or cytoprotective agent.

As shown in Example 1 and FIGS. 6-8, platelets and/or megakaryocytes express GLP-1R. Accordingly, in one embodiment, the GLP-1R agonists described herein bind transmembrane GLP-1R located on platelet or megakaryocyte cell membranes. In one embodiment, the GLP-1R agonists described herein increase intracellular cAMP levels in megakaryocytes and/or platelets. A skilled person would readily be able to identify GLP-1R agonists useful for the methods described herein that increase intracellular cAMP levels in megakaryocytes and/or platelets such as by using the assays described in Example 1.

In one embodiment, the methods and uses described herein are useful for inhibiting platelet aggregation in a subject diagnosed with, or suspected of having, cardiovascular disease. In one embodiment, the methods and uses described herein are useful for inhibiting platelet aggregation in a subject who has not been diagnosed with, or suspected of having, Type 2 diabetes. In a preferred embodiment, the methods and uses described herein are useful for inhibiting platelet aggregation in a subject at risk of a thrombotic event. For example, in one embodiment the methods and uses described herein are for inhibiting platelet aggregation in a subject who has previously had a thrombotic event such as a myocardial infarction or stroke. In one embodiment, the methods and uses described herein are for inhibiting platelet aggregation in a subject who has previously had an atherothrombotic event and/or has been diagnosed with atherosclerotic plaques. Atherosclerotic plaque rupture can lead to thrombosis, and ultimately myocardial or cerebral infarction. The use or administration of a GLP-1R agonist as described herein for inhibiting platelet aggregation can reduce the likelihood or severity of thrombus formation in a subject with atherosclerotic plaques. For example, in one embodiment, the methods and uses described herein for inhibiting platelet aggregation are for use in a subjects with type IV, V or VI plaque lesions according to the American Heart Association Guidelines (Stary et al. 1995).

In one embodiment, the methods and uses described herein are for inhibiting platelet aggregation in a subject with dyslipidemia. In one embodiment, the methods and uses described herein are useful for inhibiting platelet aggregation in a subject with low HDL cholesterol, high LDL cholesterol and high triglyceride levels.

In one embodiment, the methods and uses described herein are for inhibiting platelet aggregation in a subject at risk of thrombosis. For example, in one embodiment the subject has atrial fibrillation, or a diffuse coronary, cerebral or peripheral vascular disease. In one embodiment the subject has chronic renal failure and/or is on dialysis. In some embodiments, the subject has other symptoms of cardiovascular disease or atherosclerotic plaques.

In one embodiment, the methods and uses described herein are for inhibiting platelet aggregation in a subject intolerant or unresponsive to other antiplatelet agents. In one embodiment, the subject is intolerant or unresponsive to aspirin, ADP receptor antagonists, or thrombin inhibitors for inhibiting platelet aggregation.

In one embodiment, the methods and uses described herein are for inhibiting platelet aggregation in a subject with diabetes, obesity and/or hypertension.

In one embodiment, the methods and uses described herein are for inhibiting platelet aggregation in a subject who is experiencing a thrombotic event such as a myocardial infarction or stroke. For example, the GLP-1R agonist described herein can be administered to a subject when the subject is first identified as having a myocardial infarction or stroke such as during a period thrombosis and/or ischemic. Optionally, the methods and uses described herein are for inhibiting platelet aggregation prophylactically such as to prevent recurrence of atherothrombotic or thrombotic event. In one embodiment the GLP1-R agonists described herein are used in or administered to a subject who has previously had a thrombotic event such as a myocardial infarction or stroke.

Optionally, the methods and uses described herein for inhibiting platelet aggregation comprise the administration or use of a GLP-1R agonist in a subject with a plurality of the conditions described herein, such as dyslipidemia and atherosclerotic plaques. In one embodiment, the methods and uses described herein are for inhibiting platelet aggregation in a subject with diabetes and a high risk of thrombosis.

In one embodiment, the methods involve administering to a subject in need thereof a GLP-1R agonist or a composition comprising a GLP-1R agonist as described herein. The administration or use of a GLP-1R agonist for the prevention of platelet aggregation can be in vivo and/or ex vivo. In an embodiment, the amount of the agonist or composition used or administered to a subject is a therapeutically active amount at dosages and for periods of time necessary to achieve the desired result, namely the inhibition of platelet aggregation. In one embodiment, the amount of the agonist administered to the subject is sufficient to inhibit platelet aggregation, but does not completely abolish platelet aggregation activity in the subject. In one embodiment, the amount of the agonist or composition used or administered to a subject may be sufficient to prevent the formation of thrombosis, thereby helping to prevent ischemic events. For example, a therapeutically active amount of an agonist or composition may vary according to factors such as the disease state, age, sex, and weight of the individual, and the ability of the substance to elicit a desired response in the individual. Dosage regimes may be adjusted to provide the optimum therapeutic or prophylactic response. Optionally, the agonists described herein may be administered by recombinant expression of nucleic acids encoding for the peptide in the subject, such as by methods of gene therapy.

For example, in one embodiment, a GLP-1R agonist is used, formulated for use or administered to a subject at a dose of about 0.05 nmol/kg to about 100 nmol/kg. In one embodiment, a GLP-1R agonist is used, formulated for use or administered to a subject at a dose of about 0.1 nmol/kg to about 80 nmol/kg Optionally, the methods and uses described herein involve the use, formulation for use or administration to the subject a dose of the GLP-1R agonist of about 1 nmol/kg to about 60 nmol/kg. In one embodiment, the GLP-1R agonist is used or administered at a dose of about 10 nmol/kg to about 50 nmol/kg. In one embodiment, the GLP-1R agonist is Exendrin-4.

In one embodiment, the methods described herein involve the use or administration of a GLP1R agonist as a prophylactic for the prevention of tissue damage following a thrombotic event. Optionally the methods and uses described herein involve the administration or use of a GLP-1R agonist or compositions comprising a GLP-1R agonist after an thrombotic event has been detected in a subject. In some embodiments the peptide or composition may be used, formulated for use, or administered to the subject on a regular dosing schedule, such as about every day, every 2 days, every 3 days every 4 days, every 5 days, every 6 days, or every week. In some embodiments the peptide or composition may be used, formulated for use or administered to the subject on a regular dosing schedule such as every 10 days, every 2 weeks, every 3 weeks or every month etc.

The following non-limiting examples are illustrative of the present disclosure.

### EXAMPLES

### Example 1

### GLP-1 and GLP-1R Agonists Inhibit Thrombin-Induced Human Platelet Aggregation

### Materials and Methods

Cloning/Sequencing:

RT-PCR was performed on RNA from MEG-01 cells with primers designed to yield full-length human GLP-1R cDNA. PCR products were cloned and sequenced.

cAMP Assay:

An EIA kit (Cayman Chemicals) was employed to measure intracellular cAMP levels. MEG-01 cells were incubated for 15 min with 100 pM to 100 nM GLP-1 or Exendin-4. 3-isobutyl-1-methylxanthine (IBMX) was added to each well at a concentration of 0.5 μM and allowed to incubate for 10 min at 37° C. to inhibit phosphodiesterase activity. As a positive control for cAMP generation in MEG-01 cells, PGI2 was added at a concentration of 0.02 ng/ml to control wells and incubated for 10 min at 37° C. PBS was used as a negative control. After incubation, cells were lysed by adding EDTA to a concentration of 10 mM. Lysed cells were transferred to 1.5 ml eppendorf tubes and boiled for 5 min at 95° C. Samples were spun down at 10000 xg for 15 min at 4° C. Supernatant was removed and frozen at −80° C. The cAMP EIA kit assay was then carried out according to manufacturer's instructions.

Aggregation:

Human gel-filtered platelets were incubated with GLP-1(7-36), GLP-1(9-36), or Exendin-4 at different concentrations (100 pM-100 nM) vs. PBS controls for 15 min at 37° C. Platelets were then stimulated with thrombin and aggregation was measured using an aggregometer.

### Results

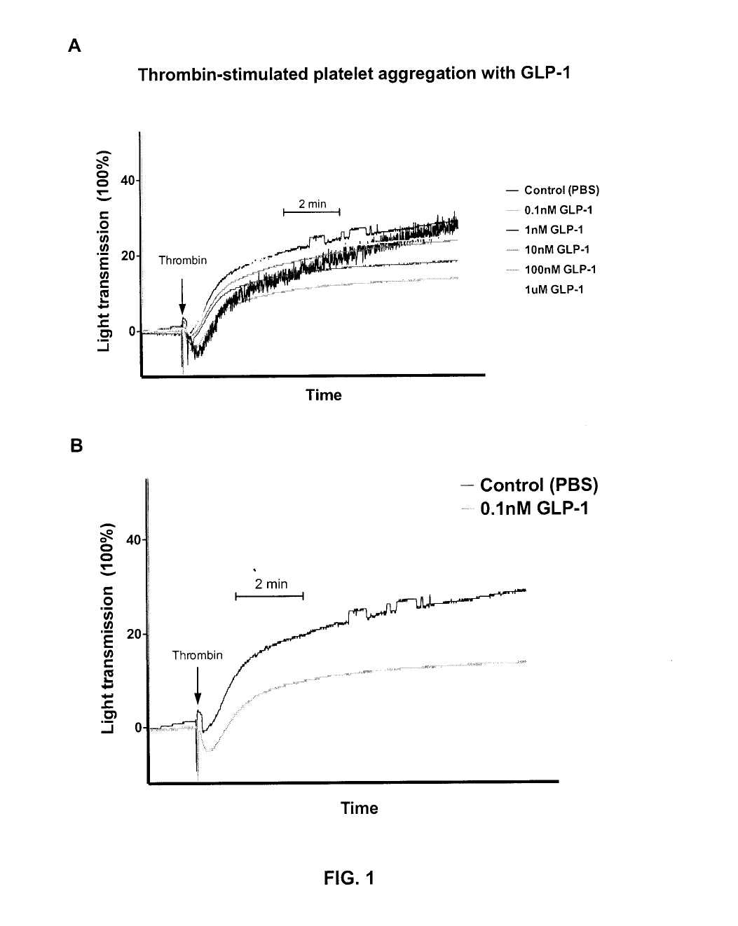

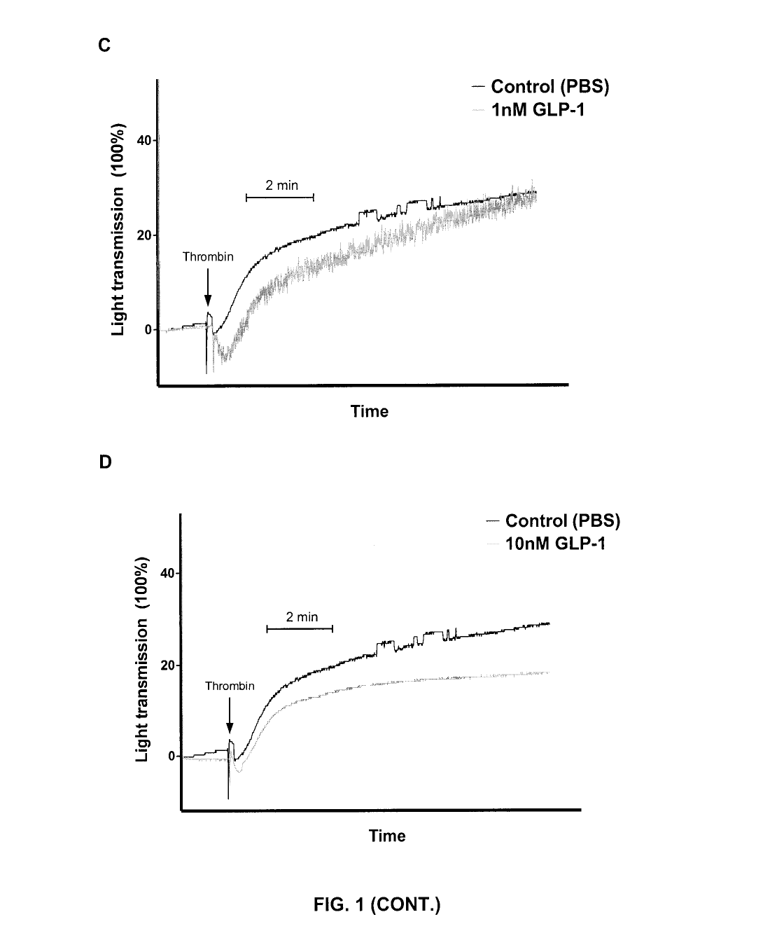

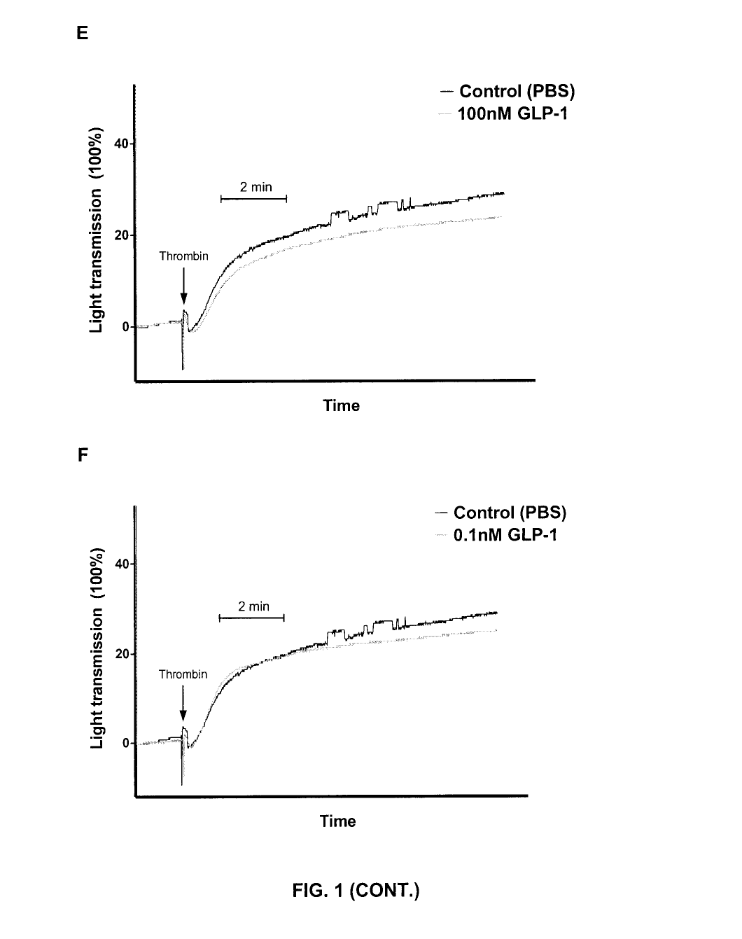

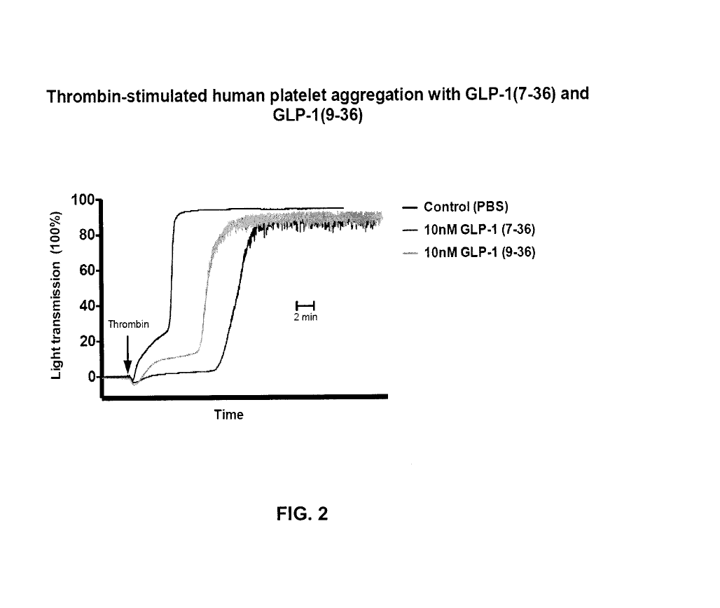

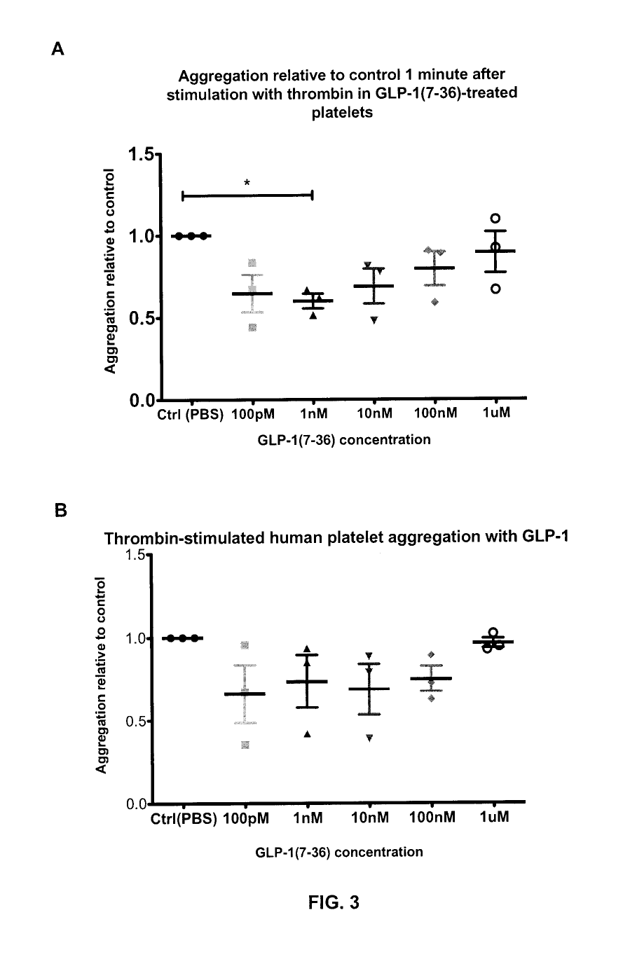

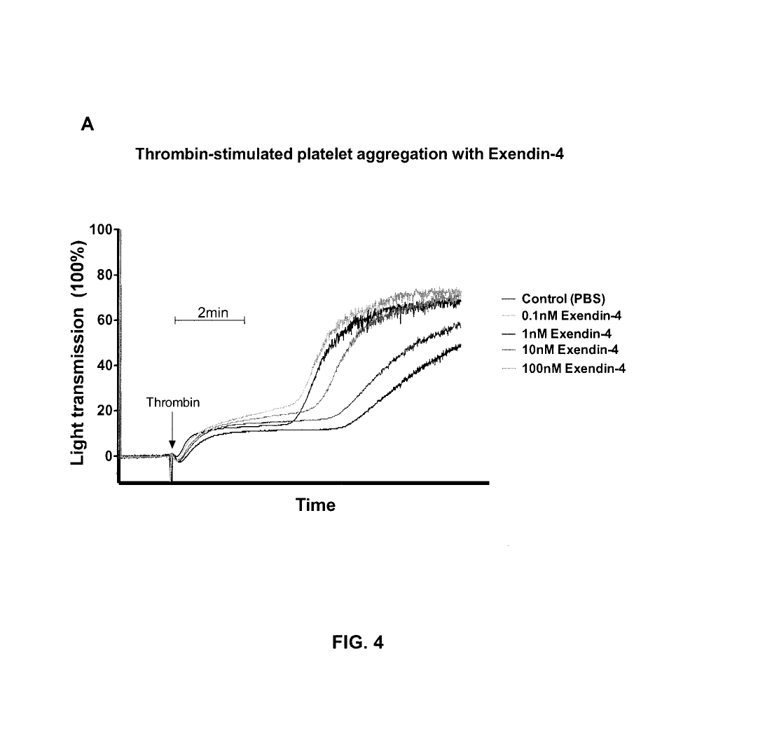

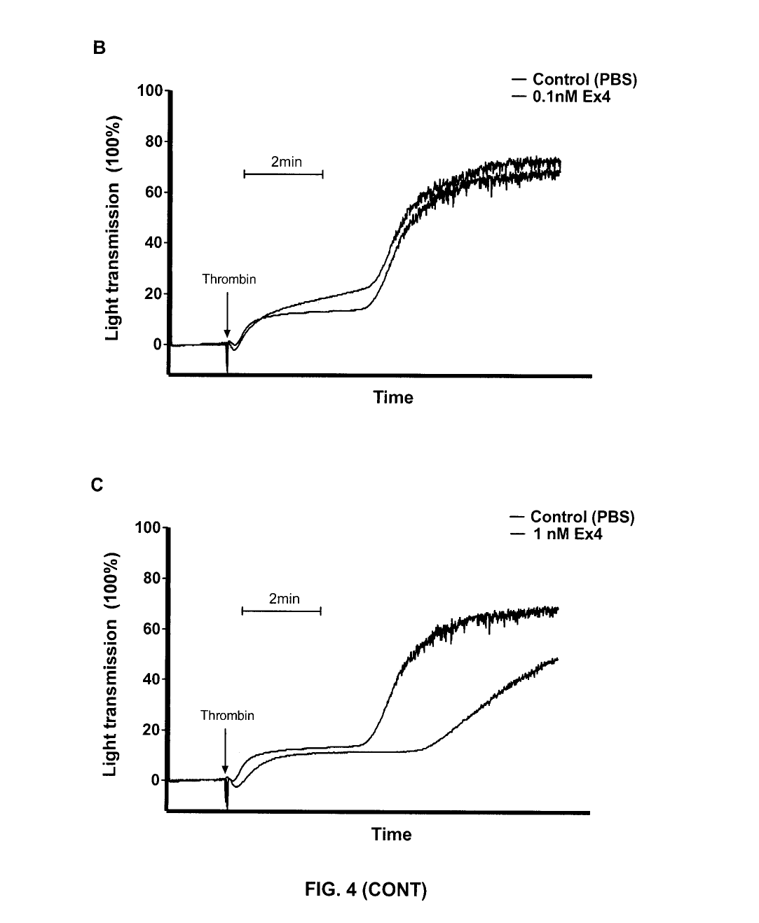

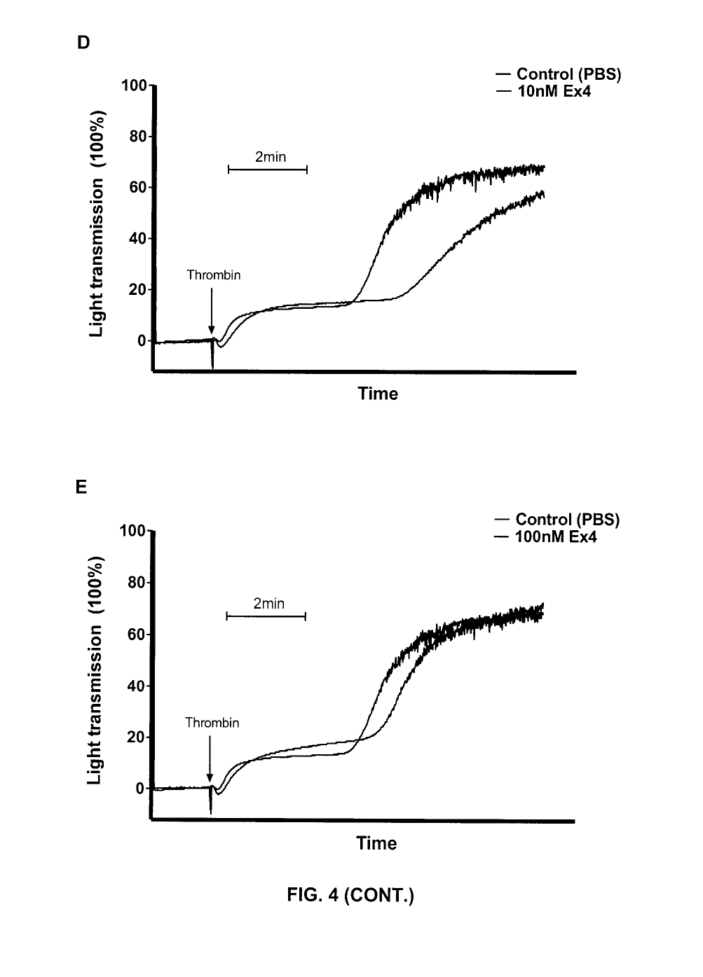

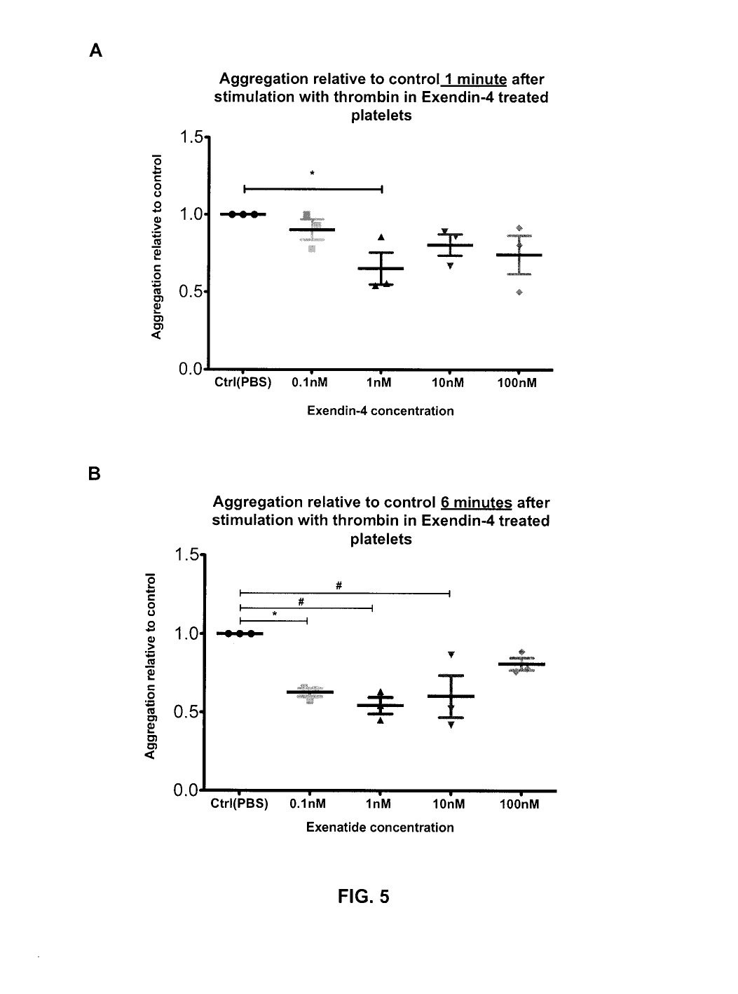

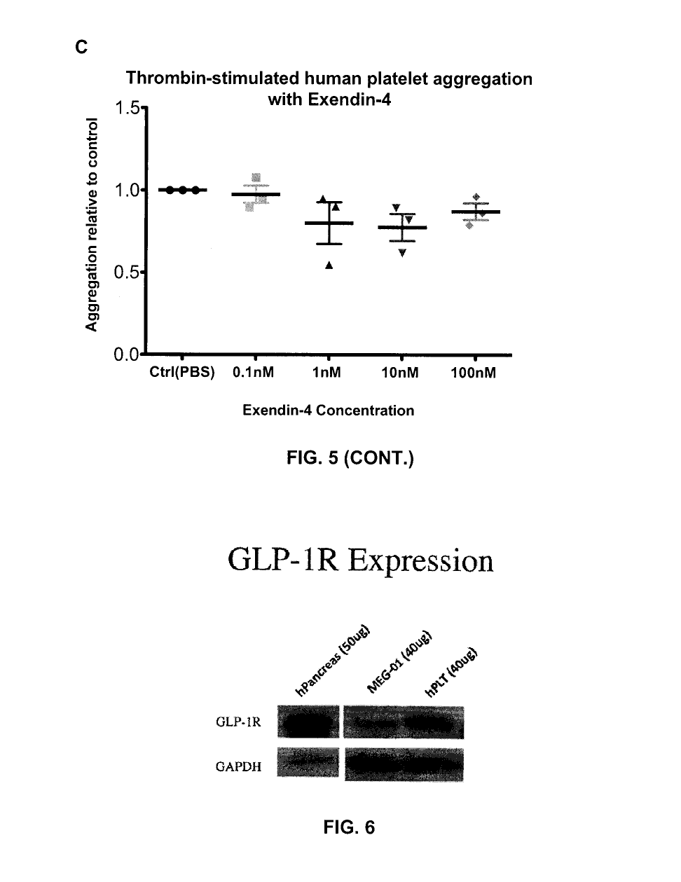

To investigate potential anti-thrombotic effects of GLP-1 and related analogs, GLP-1(7-36)^{NH2}, the most abundant endogenous GLP-1, and the commercially available GLP-1 analog exenatide were tested in thrombin-induced platelet aggregation assays using freshly isolated gel-filtered human platelets (3×10⁸/ml) in normoglycemic conditions (5.5 mM glucose). As shown in FIGS. 1 and 4, incubation with GLP-1 (10⁻¹⁰ to 10⁻⁶ M) or exenatide (5×10⁻¹⁰ to 10⁻⁸ M) for 15 min at 37° C. delayed and reduced platelet aggregation as compared to PBS-treated controls. GLP-1 (10⁻⁹M) resulted in a 60% decrease in aggregation 1 min after thrombin stimulation (FIG. 3). As shown in FIG. 2, the GLP-1 metabolite GLP-1(9-36)^{NH2} also inhibited human platelet aggregation, but to a lesser extent than GLP-1(7-36)^{NH2}. The GLP-1R agonist exenedin-4 also reduced and delayed thrombin induced platelet aggregation in a dose-dependent manner relative to PBS-treated controls (FIG. 4).

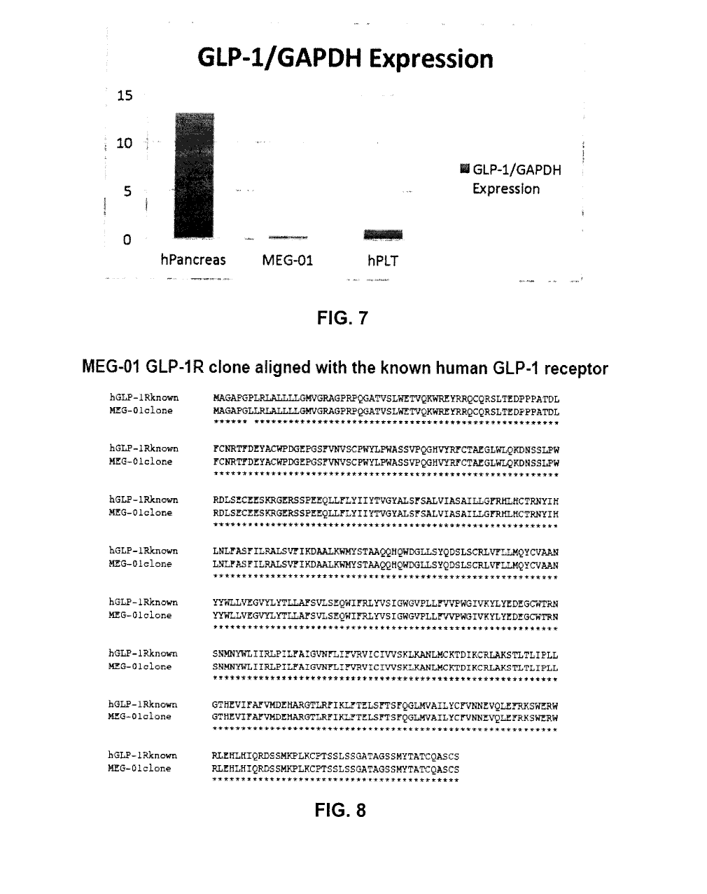

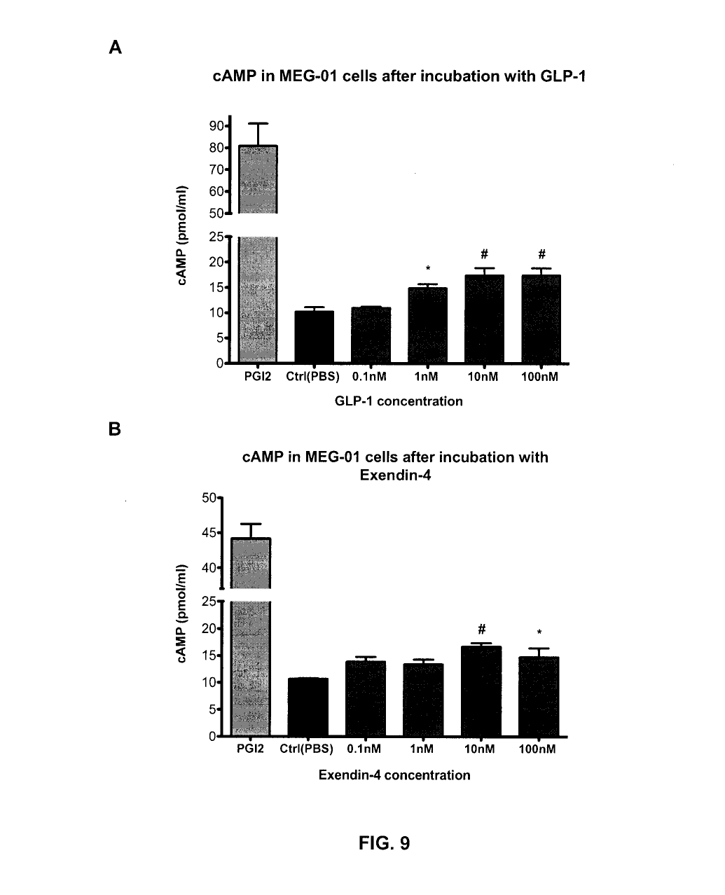

To investigate whether this effect is mediated by the GLP-1 receptor (R), whole cell lysates (40 μg) from gel-filtered human platelets and the human megakaryocyte (MGK) cell line MEG-01 were probed with a polyclonal anti-GLP-1R Ab (1:1250; Santa Cruz). Although Western blot showed bands (˜56 kD) consistent with the known GLP-1R in both platelets and MEG-01 cells (FIG. 6), GAPDH-normalized expression levels of GLP-1R were ˜13 and ˜45 fold lower in these cells than in human pancreas (FIG. 7). Given the limited specificity of Abs raised against GPCRs, RT-PCR was performed on RNA from MEG-01 cells with primers designed to yield full-length human GLP-1R cDNA. PCR products were cloned and sequenced, revealing inserts identical to GLP-1R except for a known Pro→Leu polymorphism at amino acid position 7 (FIG. 8). As the GLP-1 receptor is known to be adenylate cyclase-coupled, cAMP assays were also performed (EIA kit, Cayman Chemicals). MEG-01 cells incubated for 15 min with 10⁻⁷ M GLP-1 showed a ˜60% increase in intracellular cAMP, an inhibitor of platelet activation, as compared to untreated controls (FIG. 9A); while the cAMP response to prostacyclin PGI2 was much greater (positive control; 16 fold). MEG-01 cells incubated with the GLP-1R agonist exendin-4 also resulted in an increase in intracellular cAMP. This suggests that the GLP-1R on MEG-01 cells is functional and that the increase in cAMP levels is a result of GLP-1R stimulation and not a receptor independent effect of GLP-1 or GLP-1 metabolites.

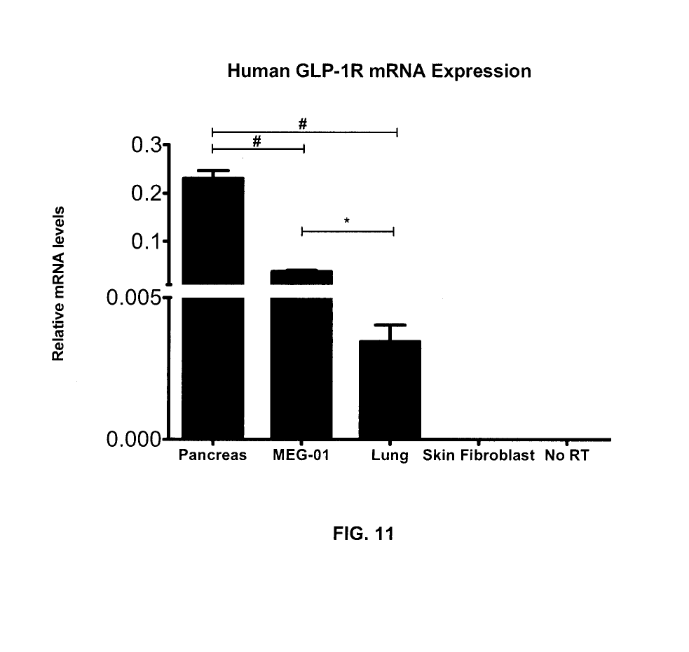

Quantitative RT-PCR was also performed on a series of human tissues samples to confirm GLP-1R mRNA expression. As shown in FIG. 11, GLP-1R mRNA levels were highest in pancreas followed by the megakaryocyte cell line MEG-01 which was observed to express more GLP-1R than lung. Little or no expression was observed in skin fibroblasts.

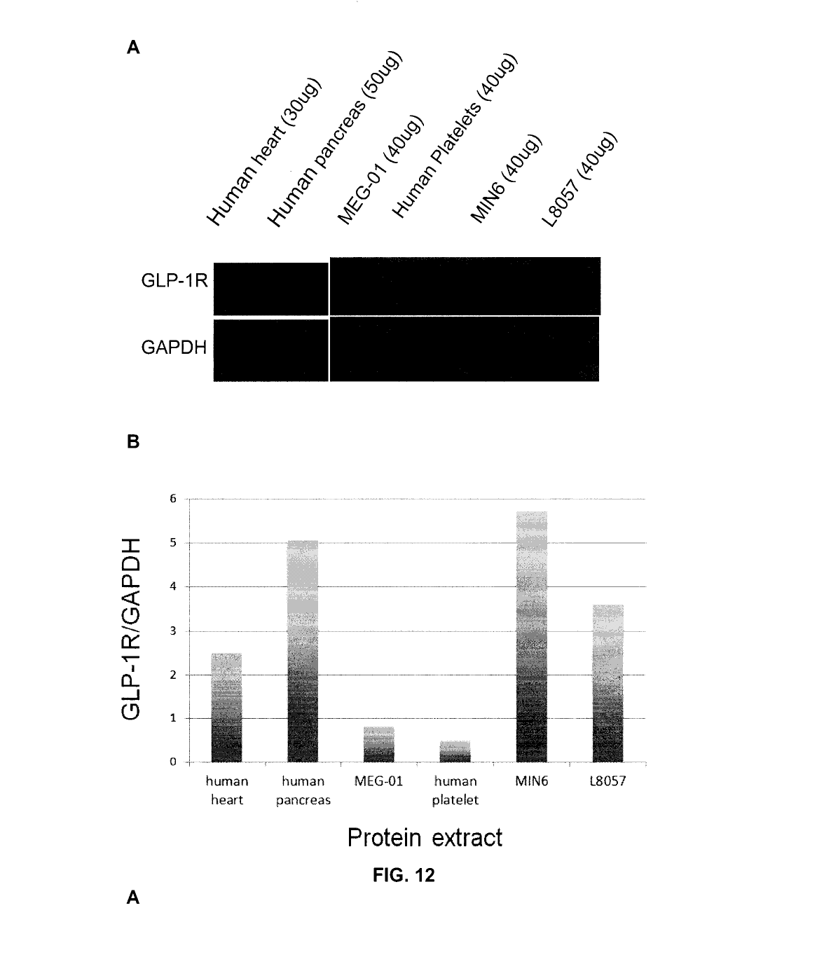

Further investigation of GLP-1R expression using Western blots in a variety of cell types including human heart, pancreas, MEG-01 cells, platelets, MIN6 cells and L8057 cells is shown in FIG. 12. A significant amount of GLP-1R expression was observed in human heart, MEG-01 cell and human platelets.

Human platelets and megakaryocytes therefore express low levels of functional GLP-1R. GLP-1 induced small increases in cAMP in megakaryocytes and both GLP-1 and exenatide inhibited thrombin-induced platelet aggregation. These data demonstrate that GLP-1R agonists possess anti-platelet effects and that the activation of GLP-1R is useful for inhibiting platelet aggregation.

### Example 2

### Human Megakaryocytes and Platelets do not Exhibit DPP4 Activity

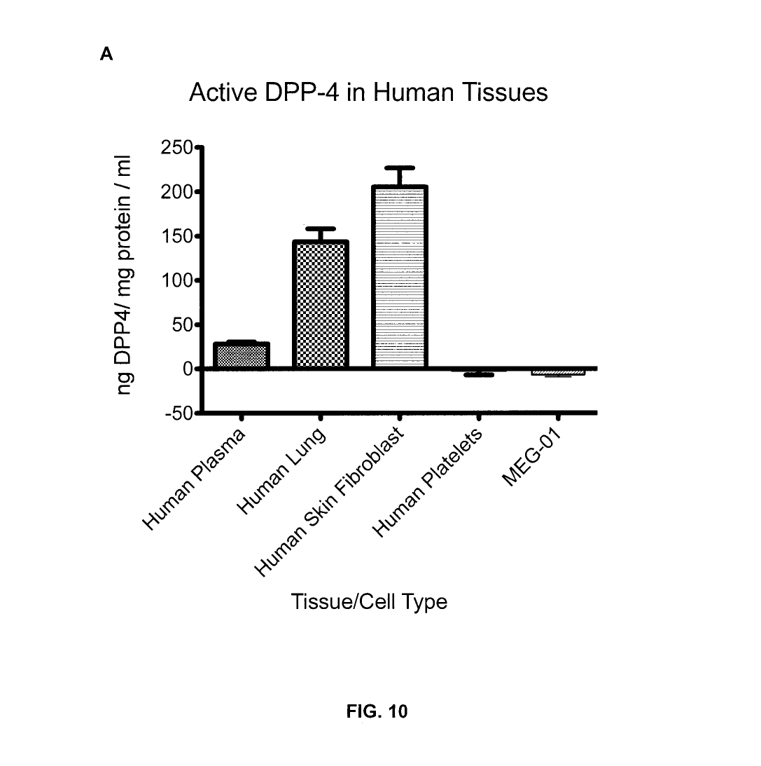

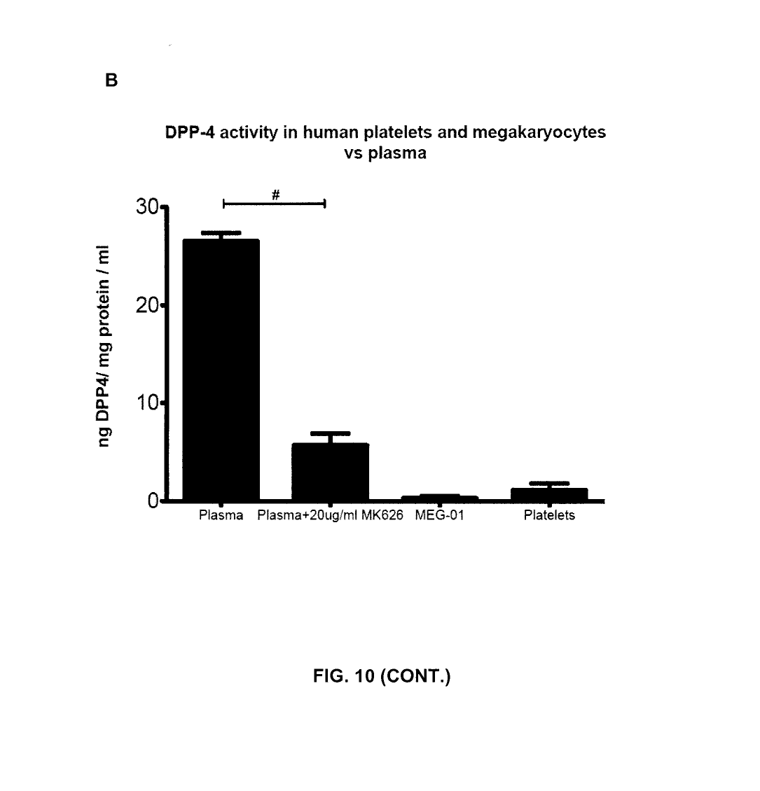

DPP-IV is an aminopeptidase expressed on many tissues throughout the body as a membrane spanning protein, and is also found in the plasma in a soluble form. DPP-IV acts as an enzyme to cleave peptides that have a proline or an alanine in the penultimate position at the N-terminal. GLP-1 has an alanine in this position, and is therefore a substrate for DPP-IV. Dipeptidyl peptidase-4 (DPP4) activity was assessed in different human cell types using the CBA085 Innozyme™ DPP4 Immunocapture Activity Assay. As shown in FIG. 10A, human platelets and megakaryocytes do not appear to exhibit any DPP4 activity. Aggregation assays were carried out using gel-filtered platelets such that there was not expected to be any soluble DPP-IV in the assay. The results of this assay show that MEG-01 cells do not harbor any detectable levels of endogenous DPP-IV activity, and expression on platelets appears to be negligible (FIG. 10B). Human plasma was used as a positive control, and human plasma treated with MK626, a sitagliptin analogue, was used as a technical control for the assay. MK626 was able to significantly inhibit DPP-IV activity in human plasma (n=3, p<0.01) (FIG. 10B). Given that platelets do not harbour DPP-IV activity, the inhibitory effect of sitagliptin on platelet aggregation reported by Gupta et al. (2012) must be a result of a direct inhibition by sitagliptin and not a DPP-IV or GLP-1 mediated response.

### Example 3

### Exendin-4 Inhibits Thrombus Formation In Vivo

An in vivo mouse cremaster arteriolar thrombosis model was used to investigate the effects of the GLP-1R agonist exendin-4. Adult mice were anesthetized and a tracheal tube inserted to facilitate breathing. Antibodies and anesthetic reagent (pentobarbital; Abbott Laboratories, Toronto, ON; 0.05 mg/kg) were administered by a jugular vein cannula. The cremaster muscle was prepared under a dissecting microscope and superfused throughout the experiment with preheated bicarbonate-buffered saline. Platelets were labelled by injecting an Alexa 660-conjugated anti-GP1b antibody. Multiple independent upstream injuries were performed on a cremaster arteriole with the use of an Olympus BX51WI microscope with a pulsed nitrogen dye laser. The dynamic accumulation of fluorescently labeled platelets within the growing thrombus was captured and analyzed using Slidebook software (Intelligent Imaging Innovations). Five minutes before injury, mice were given a high dose (60 nmol/kg) exendin-4 through the jugular vein. The mouse model and doses of Exendin-4 were otherwise similar to those described in Reheman et al. 2005; Reheman et al. 2009; Falati et al. 2003; Arakawa et al. 2010; Goto et al. 2011; and Hsieh et al. 2010, incorporated by reference herein in their entirety.

### Results

As shown in FIG. 13, a dose of 60 nmol/kg of the GLP-1R agonist Exendin-4 was observed to inhibit thrombus formation. GLP-1R agonists are therefore also expected to be useful inhibiting platelet aggregation and thrombus formation in humans.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Arakawa et al., Inhibition of monocyte adhesion to endothelial cells and attenuation of atherosclerotic lesion by a glucagon-like peptide-1 receptor agonist, exendin-4. Diabetes, 2010 59(4): p. 1030-7.
Dunne et al. Cadherin 6 Has a Functional Role in Platelet Aggregation and Thrombus Formation. Arterioscler Thromb Vasc Biol. 2012; 32:1724-1731.
Falati et al., Real-time in vivo imaging of platelets, tissue factor and fibrin during arterial thrombus formation in the mouse. Nature medicine, 2002. 8(10): p. 1175-81.
Goto et al., Exendin-4 a glucagon-like peptide-1 receptor agonist, reduces intimal thickening after vascular injury. Biochem Biophys Res Commun, 2011 405(1): p. 79-84.
Gupta et al., Sitagliptin: anti-platelet effect in diabetes and healthy volunteers. Platelets. 2012; 23(8):565-70.
Hsieh et al., The glucagon-like peptide 1 receptor is essential for postprandial lipoprotein synthesis and secretion in hamsters and mice. Diabetologia, 2010 53(3): p. 552-61.
Monami et al., Dipeptydil peptidase-4 inhibitors in type 2 diabetes: a meta-analysis of randomized clinical trials. Nutrition, metabolism, and cardiovascular diseases: NMCD, 2010. 20(4): p. 224-35
Monami et al., Glucagon-like peptide-1 receptor agonists and cardiovascular events: a meta-analysis of randomized clinical trials. Experimental Diabetes Research, 2011. p. 215764
Ni et al. Persistence of platelet thrombus formation in arterioles of mice lacking both von Willebrand factor and fibrinogen J. Clin. Invest. 106:385-392 (2000).
Reheman et al., Vitronectin stabilizes thrombi and vessel occlusion but plays a dual role in platelet aggregation. Journal of thrombosis and haemostasis: JTH, 2005. 3(5): p. 875-83.
Reheman et al., Plasma fibronectin depletion enhances platelet aggregation and thrombus formation in mice lacking fibrinogen and von Willebrand factor. Blood, 2009. 113(8): p. 1809-17.
Scirica et al., Saxagliptin and Cardiovascular Outcomes in Patients with Type 2 Diabetes Mellitus. N Engl J Med. 2013 Oct. 3; 369(14):1317-26
Stary et al., A Definition of Advanced Types of Atherosclerotic Lesions and a Histological Classification of Atherosclerosis. Circulation. 1995; 92: 1355-1374
Turnbull et al., Intensive glucose control and macrovascular outcomes in type 2 diabetes. Diabetologia, 2009. 52(11): p. 2288-98

1. A method for inhibiting platelet aggregation in a subject comprising administering to the subject a Glucagon-Like Peptide-1 receptor (GLP-1R) agonist.

2. The method of claim 1, wherein the GLP-1R agonist is exogenous GLP-1(7-36) or GLP-1(9-36).

3. The method of claim 1, wherein the GLP-1R agonist is a GLP-1 analog.

4. The method of claim 3, wherein the GLP-1 analog is exenatide.

5. The method of claim 1, wherein the GLP-1R agonist is selected from exenatide, liraglutide, albiglutide, taspoglutide and lixisenatide.

6. The method of claim 1, wherein the GLP-1R agonist binds to GLP-1R on platelets or megakaryocytes.

7. The method of claim 1, wherein the GLP-1R agonist increases intracellular cAMP levels in megakaryocytes and/or platelets.

8. The method of claim 1, wherein the subject has cardiovascular disease.

9. The method of claim 1, wherein the subject does not have Type 2 diabetes.

10. The method of claim 1, wherein the subject has previously had a thrombotic event.

11. The method of claim 10, wherein the thrombotic event is a myocardial infarction or stroke.

12. The method of claim 1, wherein the subject has atherosclerotic plaques.

13. The method of claim 1, wherein the GLP-1R agonist is administered by injection.

14.-26. (canceled)

27. The method of claim 1, for the prevention or treatment of thrombotic cerebrovascular or cardiovascular disease.

28. The method of claim 27, wherein the subject has Type 2 diabetes.

29. The method of claim 28, wherein the subject has a fasting glucose level of 126 mg/dL or higher.

30. The method of claim 27, wherein the subject previously had a thrombotic event.

31. The method of claim 30, wherein the thrombotic event is a myocardial infarction or stroke.

32. The method of claim 27, wherein the subject has atherosclerotic plaques.

33. The method of claim 1, wherein the GLP-1R agonist is co-administered with an anticoagulant, thrombolytic agent, anti-inflammatory and/or cytoprotective agent.


SEQUENCE LISTING (5 sequences)

SEQ ID NO: 1 - protein, 463 aa, Homo sapiens
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 2 - protein, 463 aa, Homo sapiens [sequence omitted]

SEQ ID NO: 3 - protein, 30 aa, Artificial Sequence
  Synthetic construct (GLP-1(7-36))
HAEGTFTSDVSSYLEGQAAKEFIAWLVKGR

SEQ ID NO: 4 - protein, 28 aa, Artificial Sequence
  Synthetic construct (GLP-1(9-36))
EGTFTSDVSSYLEGQAAKEFIAWLVKGR

SEQ ID NO: 5 - protein, 39 aa, Artificial Sequence
  Synthetic construct (exendin-4)
HGEGTFTSDLSKQMEEEAVRLFIEWLKNGGPSSGAPPPS